(19) United States
(12) Patent Application Publication
(10) Pub. No.: US 2012/0258937 A1
(43) Pub. Date: Oct. 11, 2012

(54) Targeting Estrogen Receptors in the Treatment of Lymphangioleiomyomatosis

(75) Inventors: Elizabeth P. Henske, Cambridge, MA (US); Jane Yu, Brighton, MA (US)

(73) Assignee: Fox Chase Cancer Center, Philadelphia, PA (US)

(21) Appl. No.: 13/442,249

(22) Filed: Apr. 9, 2012

Related U.S. Application Data
(60) Provisional application No. 61/473,302, filed on Apr. 8, 2011.

(51) Int. Cl. A61K 31/56; C12N 5/077; A61P 11/00; A61K 31/65

(52) U.S. Cl. 514/154; 514/182; 435/375

(57) ABSTRACT

Methods for inhibiting estrogen hormone-induced pulmonary metastasis of smooth muscle cells that are capable of pulmonary metastasis comprise antagonizing the estradiol receptor on the smooth muscle cells such that pulmonary metastasis of the cells is inhibited.

### CROSS REFERENCE TO RELATED APPLICATIONS

This application claims priority to U.S. Provisional Application No. 61/473,302 filed on Apr. 8, 2011, the contents of which are incorporated by reference herein, in their entirety and for all purposes.

### STATEMENT OF GOVERNMENT SUPPORT

The inventions described herein were made, in part, with funds obtained from the National Heart Lung and Blood Institute, Grant Nos. HL31147 and HL098216. The U.S. government may have certain rights in these inventions.

### FIELD OF THE INVENTION

The invention relates generally to the field of lymphangioleiomyomatosis treatment. More particularly, the invention relates to compositions and methods for antagonizing the estradiol receptor such that pulmonary metastasis of smooth muscle cells is inhibited.

### BACKGROUND OF THE INVENTION

Various publications, including patents, published applications, technical articles and scholarly articles are cited throughout the specification. Each of these cited publications is incorporated by reference herein, in its entirety and for all purposes.

Lymphangioleiomyomatosis (LAM) is a progressive pulmonary disease which affects almost exclusively women. LAM is characterized pathologically by widespread proliferation of abnormal smooth muscle cells and by cystic changes within the lung parenchyma. LAM occurs in 30-40% of women with tuberous sclerosis complex (TSC). LAM can also occur in women who do not have germline mutations in TSC1 or TSC2 (sporadic LAM). Inactivating mutations of both alleles of the TSC1 or TSC2 genes have been found in LAM cells from both TSC-LAM and sporadic LAM patients. Astrinidis A et al. (2000) J. Med. Genet. 37:55-57 and Strizheva G D et al. (2001) Am. J. Respir. Crit. Care Med. 163:253-258.

The protein products of the TSC1 and TSC2 genes, hamartin and tuberin, respectively, form heterodimers that inhibit the small GTPase Rheb (Ras homologue enriched in brain), via tuberin's highly conserved GTPase activating domain. Loss of tuberin or hamartin leads to hyperactivation of the mammalian target of Rapamycin complex 1 (mTORC1), which has been observed in LAM cells.

Tumor metastasis is a multistep event involving the tumor cells dissemination from the primary tumor to seed at remote tissue to form metastatic lesions. One of the initial steps of metastasis is the degradation of the basement membrane. Matrix metalloproteinases (MMPs) are involved in the degradation of extracellular matrix (ECM), thereby facilitating tumor cell invasion, metastasis and angiogenesis. Elevated levels of MMP1, MMP2, MMP9, MMP14, and cathepsin K have been observed in LAM lung nodules.

Despite recent progress in LAM research, there remains a need for improved therapeutic strategies. Currently, there are no U.S. FDA-approved drugs for treatment of end-stage LAM. Treatment with Rapamycin, an mTORC1 inhibitor, can stabilize lung function in LAM, but lung function continues to decline when the drug is discontinued (McCormack F X, et al. (2011) N. Engl. J. Med. 364:1595-1606). The only proven treatment for end-stage LAM is lung transplantation, which carries significant one-year mortality and after which LAM can recur in the transplanted lungs.

### SUMMARY OF THE INVENTION

The invention features methods for treating lymphangioleiomyomatosis (LAM) in a subject in need thereof. In general, the methods comprise administering to the subject an effective amount of Fulvestrant, a pharmaceutically acceptable salt of Fulvestrant, a composition comprising an effective amount of Fulvestrant and a pharmaceutically acceptable carrier, or a composition comprising an effective amount of a pharmaceutically acceptable salt of Fulvestrant and a pharmaceutically acceptable carrier. Optionally, the methods may comprise administering to the subject an effective amount of Doxycycline. The subject is preferably a human being. The human being may be female or male. The human being may have tuberous sclerosis complex-associated LAM. The human being may have at least one germline mutation in the gene encoding tuberous sclerosis protein 2 (TSC2) or in the gene encoding tuberous sclerosis protein 1 (TSC1). The human being may not have any germline mutation in either TSC1 or TSC2, e.g., the human being may have sporadic LAM.

The invention also features methods for inhibiting estrogen hormone-induced pulmonary metastasis of smooth muscle cells. In some aspects, the smooth muscle cells have at least one mutation in the gene encoding tuberous sclerosis protein 2 (TSC2) or have at least one mutation in the gene encoding tuberous sclerosis protein 1 (TSC1). In some aspects, the smooth muscle cells do not have any germline mutation in either TSC1 or TSC 2. The smooth muscle cells may express one or more of melanoma glycoprotein 100 and the HMB 45 antigen. The methods may also inhibit metastasis of cells in any organs distal from the locus of the cells in their non-LAM state. In general, the methods comprise antagonizing an estrogen receptor on the smooth muscle cells such that metastasis of the cells is inhibited. In preferred aspects, pulmonary metastasis of the cells is inhibited.

The estrogen hormone may be estradiol. Thus, for example, the methods may inhibit estradiol-induced pulmonary metastasis of smooth muscle cells.

Antagonizing the estrogen receptor may comprise contacting the smooth muscle cells with an estrogen receptor antagonist in an amount effective to antagonize the estrogen receptor. In aspects where the estrogen receptor is the estradiol receptor, antagonizing the estradiol receptor may comprise contacting the smooth muscle cells with an estradiol receptor antagonist in an amount effective to antagonize the estrogen receptor. An estradiol receptor antagonist may comprise Fulvestrant or a pharmaceutically acceptable salt thereof, or may comprise a composition comprising a pharmaceutically acceptable carrier and Fulvestrant or a pharmaceutically acceptable salt thereof.

In some preferred aspects, the methods are carried out in vivo. For example, antagonizing the estrogen receptor may comprise administering to a subject in need thereof an estrogen receptor antagonist in an amount effective to antagonize the estrogen receptor. In aspects where the estrogen receptor is the estradiol receptor, antagonizing the estradiol receptor may comprise administering to the subject an estradiol receptor antagonist in an amount effective to antagonize the estrogen receptor. An estradiol receptor antagonist may comprise Fulvestrant or a pharmaceutically acceptable salt thereof, or may comprise a composition comprising a pharmaceutically acceptable carrier and Fulvestrant or a pharmaceutically acceptable salt thereof. Administering an antagonist may facilitate contact of the smooth muscle cells with the antagonist.

The subject is preferably a human being. The human being may be female or male. The human being may have tuberous sclerosis complex-associated LAM. The human being may have at least one germline mutation in the gene encoding tuberous sclerosis protein 2 (TSC2) or in the gene encoding tuberous sclerosis protein 1 (TSC1). The human being may not have any germline mutation in either TSC1 or TSC2, e.g., the human being may have sporadic LAM.

The invention also features kits for treating lymphangioleiomyomatosis (LAM). The kits may comprise a composition comprising a pharmaceutically acceptable carrier and an amount of Fulvestrant or a pharmaceutically acceptable salt thereof effective to treat LAM, and instructions for using the kit in a method for treating LAM, or instructions for using the kit in a method for inhibiting estrogen hormone-induced pulmonary metastasis of smooth muscle cells, or instructions for using the kit in a method for inhibiting estrogen hormone-induced expression or biologic activity of matrix metalloproteinase 2 (MMP2).

### BRIEF DESCRIPTION OF THE DRAWINGS

FIG. 1 shows that estrogen disrupts extracellular matrix organization and reduces Type IV collagen accumulation in xenograft tumors of female and male mice. Primary tumor sections from placebo-treated and estrogen-treated female (FIG. 1A) and male (FIG. 1B) mice were stained with H & E (a & b) and type IV collagen (c & d). (Scale bar, 16 μM)

FIG. 2 shows estrogen enhances MMP2 expression in xenograft tumors from placebo-treated and estrogen-treated SCID mice. FIG. 2A shows the level of progesterone receptor (Pgr), Mmp2, and Mmp9 measured using real-time RT-PCR in primary tumors from placebo-treated (n=4) and estrogen-treated (n=4) female SCID mice. FIG. 2B shows the level of MMP2 protein measured by immunoblot analysis in primary tumors from placebo-treated (n=6) and estrogen-treated (n=7) female SCID mice, and placebo-treated (n=4) and estrogen-treated (n =4) male SCID mice. FIG. 2C shows Beta-Actin immunoblotting included as a loading control. Scatter plots show the relative levels of MMP2 normalized to β-Actin. *P<0.05, Student's t-test.

FIG. 3 shows estrogen induces MMP2 expression and activity in tuberin-null ELT3 cells. FIG. 3A shows ELT3 cells were grown in phenol red-free and serum-free media for 24 hours and then stimulated with 1 μM E2 for 0, 0.5, 2, 4, or 12 hours. FIG. 3B shows ELT3 cells were incubated with MEK1/2 inhibitor PD98059 for 30 minutes, and then treated with 10 nM E₂ for 0, 2, or 4 hours. Levels of MMP2 were determined by immunoblot analysis. Beta-actin immunoblotting was included as a loading control. Densitometry analyses showed the fold of change of MMP2. FIG. 3C shows conditioned media from ELT3 cells after 10 nM E₂ stimulation for 0, 5, 15, or 120 minutes were collected, and levels of active MMP-2 were examined using gelatin zymogram gels. FIG. 3D shows ELT3 cells were pre-incubated with 10 μM ICI182780 for 4 hours followed by 10 nM E₂ stimulation for 24 hours. Conditioned media were analyzed for MMP2 activity using gelatin zymogram gels. Densitometry analyses showed the fold of change of MMP2 activity. *p<0.05, **p<0.01.

FIG. 4 shows Doxycycline does not normalize estrogen-induced ECM disruption in TSC2-deficient xenograft tumors. ELT3 cells were subcutaneously injected into female ovariectomized mice implanted with estrogen or placebo pellets. Animals were treated with Doxycycline (0.7 mg pellet, 60-day release) starting one day post-cell inoculation. FIG. 4A shows the primary tumor area was calculated at eight-week post cell inoculation. FIG. 4B shows primary tumor sections from placebo-treated and estrogen-treated female mice were stained with H & E (a, b), Ki67 (c, d) and TUNEL (e,f). (Scale bar, 16 μM). FIG. 4C shows the level of phospho-p42/44 MAPK measured by immunoblot analysis in primary tumors from placebo-treated (n=2), estrogen-treated (n=3) and estrogen plus Doxycycline-treated (n=2) female SCID mice. Beta-Actin immunoblotting was included as a loading control. FIG. 4D shows Kaplan-Meier analysis of overall survival in mice bearing xenograft tumors. FIG. 4E shows the number of lung metastases in female mice was scored from Doxycycline plus placebo (Dox+P) (n=5) and Doxycycline plus E₂ (Dox+E₂) (n=4) mice. *p<0.05, Student's t-test.

FIG. 5 shows Fulvestrant normalizes estrogen-induced ECM disruption in TSC2-deficient xenograft tumors. ELT3 cells were subcutaneously injected into female ovariectomized mice implanted with estrogen or placebo pellets. Animals were treated with Fulvestrant (1 mg/kg/day by intramuscular injection) starting one day post-cell inoculation. FIG. 5A shows the primary tumor area calculated at eight-week post cell inoculation. FIG. 5B shows primary tumor sections from placebo plus Doxycycline-treated and estrogen plus Doxycycline-treated female mice stained with H & E (a, b), Ki67 (c, d) and TUNEL (e, f). FIG. 5C shows ERα and phospho-p42/44 MAPK. (Scale bar, 16 μM).

FIG. 6 shows Fulvestrant blocks estrogen-promoted lung metastases of tuberin-null ELT3 cells and increases the survival in vivo. ELT3 cells were subcutaneously injected into female ovariectomized mice implanted with estrogen or placebo pellets. Animals were treated with Fulvestrant (1 mg/kg/day by intramuscular injection) starting one day post-cell inoculation. FIG. 6A shows the number of lung metastases in male mice scored from placebo (P) (n=10), E₂-treated (n=9), Fulvestrant plus placebo (Ful+P) (n=5) and Fulvestrant plus E₂ (Ful+E₂) (n=4) mice. FIG. 6B shows Kaplan-Meier analysis of overall survival in mice bearing xenograft tumors. *p<0.05, Student's t-test.

### DETAILED DESCRIPTION OF THE INVENTION

Various terms relating to aspects of the invention are used throughout the specification and claims. Such terms are to be given their ordinary meaning in the art, unless otherwise indicated. Other specifically defined terms are to be construed in a manner consistent with the definition provided herein.

As used herein, the singular forms “a,” “an,” and “the” include plural referents unless expressly stated otherwise.

Subject and patient are used interchangeably. A subject may be any animal, including mammals such as companion animals, laboratory animals, and non-human primates. Human beings are preferred. Female human beings are highly preferred.

Inhibiting includes reducing, decreasing, blocking, preventing, delaying, inactivating, desensitizing, stopping, and/or downregulating.

Fulvestrant comprises the chemical formula, Formula I:

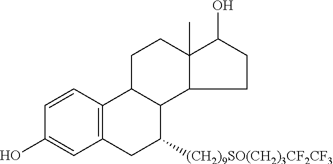

Pharmaceutically acceptable salts may be acid or base salts. Non-limiting examples of pharmaceutically acceptable salts include sulfates, methosulfates, methanesulfates, pyrosulfates, bisulfates, sulfites, bisulfites, nitrates, besylates, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, dioates, benzoates, chlorobenzoates, methylbenzoates, dinitromenzoates, hydroxybenzoates, methoxybenzoates, phthalates, sulfonates, toluenesulfonates, xylenesulfonates, pheylacetates, phenylpropionates, phenylbutyrates, citrates, lactates, γ-hydroxybutyrates, glycollates, tartrates, methanesulfonates, propanesulfonates, mandelates, and other salts customarily used or otherwise FDA-approved.

It has been observed in accordance with the invention that estradiol alters extracellular matrix (ECM) organization, increases the expression and activity of MMP2, and reduces the accumulation of ECM protein type IV collagen in tuberin-null xenograft tumors in mice. MMP2 enhancement was associated with changes of molecular components within the ECM that may be linked to the metastatic potential of TSC2-deficient cells. It has been further observed that targeting the estrogen receptor with Fulvestrant completely blocked estradiol-promoted lung metastasis, and MMP2 expression and activity, suggesting that targeting estrogen and its receptors may have therapeutic benefit against LAM. Accordingly, the invention features methods for inhibiting estrogen hormone-induced metastasis of smooth muscle cells. In some preferred aspects, pulmonary metastasis is inhibited. The methods may be carried out in vivo, in vitro, ex vivo, or in situ.

In some aspects, a method for inhibiting estrogen-induced metastasis of smooth muscle cells comprises antagonizing an estrogen receptor on the smooth muscle cells such that metastasis of the cells is inhibited. In some aspects, a method for inhibiting estrogen-induced pulmonary metastasis of smooth muscle cells comprises antagonizing an estrogen receptor on the smooth muscle cells such that pulmonary metastasis of the cells is inhibited. In some preferred aspects, the smooth muscle cells have at least one mutation in the gene encoding tuberous sclerosis protein 2 (TSC2). In some preferred aspects, the smooth muscle cells have at least one mutation in the gene encoding tuberous sclerosis protein 1 (TSC1). In some preferred aspects, the smooth muscle cells have at least one mutation in the gene encoding TSC1 and at least one mutation in the gene encoding TSC2. Preferably, the at least one mutation is in the germline gene encoding TSC2 or in the germline gene encoding TSC1. In some other preferred aspects, the smooth muscle cells do not have any mutation in the gene encoding TSC1 or in the gene encoding TSC2.

The methods may be used to inhibit any estrogen-induced metastasis, preferably pulmonary metastasis, of smooth muscle cells. The estrogen may be natural or synthetic, may be an estrogen hormone, including estradiol (E₂), estriol (E₃), or estrone (E₁), may be a phytoestrogen, may be a mycoestrogen, may be a xenoestrogen, or any combination thereof. Preferably, the methods may be used to inhibit estradiol-induced pulmonary metastasis of smooth muscle cells.

Antagonizing the estrogen receptor may comprise antagonizing the estradiol receptor. In some aspects, antagonizing the estrogen receptor comprises contacting the smooth muscle cells with an estrogen receptor antagonist in an amount effective to antagonize the estrogen receptor. In some aspects, antagonizing the estrogen receptor comprises contacting the estrogen receptor on the smooth muscle cells with an estrogen receptor antagonist in an amount effective to antagonize the estrogen receptor. The estradiol receptor antagonist may comprise Fulvestrant or a pharmaceutically acceptable salt thereof. The estradiol receptor antagonist may comprise a composition comprising Fulvestrant or a pharmaceutically acceptable salt thereof and a pharmaceutically acceptable carrier.

Antagonizing the estrogen receptor may comprise administering to a subject in need thereof an estrogen receptor antagonist in an amount effective to antagonize the estrogen receptor. The subject may be a mouse. The subject may be a human being, preferably a female human being. The human being may have tuberous sclerosis complex-associated LAM. The human being may have at least one germline mutation in the gene encoding TSC2. The human being may have at least one germline mutation in the gene encoding TSC1. The human being may have at least one germline mutation in the gene encoding TSC2 and at least one germline mutation in the gene encoding TSC1. The human being may not have any germline mutation in either the gene encoding TSC1 or the gene encoding TSC2. The human being may have sporadic LAM.

Mutations in the gene encoding TSC1 or TSC2 may be at any locus in the germline of each respective gene. The mutation may comprise a deletion mutation. The mutation may comprise a truncation mutation. The mutation may comprise a missense mutation. The mutation may be a missense mutation at Arg611 (exon 16), Pro1675Leu (exon 38), or an 18-bp in-frame deletion in exon 40 of TSC2. It is believed that missense mutations and large genomic deletions are more frequent in TSC2 than in TSC1. Missense mutations in TSC2 may cluster in the GTPase-activating protein (GAP) binding domain (exons 35 through 39). The TSC2 gene is located on chromosome 16p13. The TSC1 gene is located on chromosome 9q34. Allelic variants of TSC1 and TSC2 are catalogued at the Leiden Open Variation Database, see http://chromium.liacs.nl/LOVD2/TSC/home.php?select_db=TSC1 and http://chromium.liacs.nl/LOVD2/TSC/home.php?select_db=TSC2. See also Crino P B et al. (2006) N. Engl. J. Med. 355:1345-56, Astrinidis A et al. (2000) J. Med. Genet. 37:55-57, and Strizheva G D et al. (2001) Am. J. Respir. Crit. Care Med. 163:253-258.

The estrogen receptor may comprise the estradiol receptor. The estradiol receptor antagonist may comprise Fulvestrant or a pharmaceutically acceptable salt thereof. The estradiol receptor antagonist may comprise a composition comprising Fulvestrant or a pharmaceutically acceptable salt thereof and a pharmaceutically acceptable carrier.

The methods may inhibit estrogen hormone-induced pulmonary metastasis of smooth muscle cells that, for example, initiate, induce, cause, exacerbate, or facilitate LAM. The smooth muscle cells may be any type of smooth muscle cells. The smooth muscle cells may be smooth muscle cells capable of pulmonary metastasis. In some aspects, the smooth muscle cells express the melanoma glycoprotein 100 (GP100). In some aspects, the smooth muscle cells express the HMB 45 antigen. In some aspects, the smooth muscle cells express both GP100 and the HMB 45 antigen. In some aspects, the smooth muscle cells express an estrogen receptor. In some aspects, the smooth muscle cells express the estradiol receptor. In some aspects, the smooth muscle cells express one or more of GP100, the HMB 45 antigen, and an estrogen receptor, which may be the estradiol receptor.

It is believed that smooth muscle cells in LAM express higher levels of estrogen receptors than normal smooth muscle cells. It is also believed that smooth muscle cells in LAM express higher levels of estrogen receptors during the initial stages of metastasis, and that cells at their primary location (pre-metastasis) may be sensitive to estrogen. Estrogen may prolong survival of smooth muscle cells that leave their primary location as they metastasize. Yu J et al. (2010) Lymphatic Res. and Biol. 8:43-9.

The invention also features methods for inhibiting estrogen hormone-induced expression or biologic activity of matrix metalloproteinase 2 (MMP2). The methods may inhibit estrogen-hormone induced expression or biologic activity of MMP2 by a smooth muscle cell, particularly a smooth muscle cell capable of pulmonary metastasis. In some aspects, the smooth muscle cells express the melanoma glycoprotein 100 (GP100). In some aspects, the smooth muscle cells express the HMB 45 antigen. In some aspects, the smooth muscle cells express both GP100 and the HMB 45 antigen. In some aspects, the smooth muscle cells express an estrogen receptor. In some aspects, the smooth muscle cells express the estradiol receptor. In some aspects, the smooth muscle cells express one or more of GP100, the HMB 45 antigen, and an estrogen receptor, which may be the estradiol receptor. Cells at any distal sites may also respond to estradiol, thus estradiol's biologic activity may also be inhibited by the estradiol receptor antagonist. The methods may be carried out in vivo, in vitro, ex vivo, or in situ.

Inhibiting the estrogen hormone-induced expression or biologic activity of MMP2 may comprise antagonizing an estrogen receptor on the smooth muscle cells. Antagonizing the estrogen receptor may comprise antagonizing the estradiol receptor. In some aspects, antagonizing the estrogen receptor comprises contacting the smooth muscle cells with an estrogen receptor antagonist in an amount effective to antagonize the estrogen receptor. In some aspects, antagonizing the estrogen receptor comprises contacting the estrogen receptor on the smooth muscle cells with an estrogen receptor antagonist in an amount effective to antagonize the estrogen receptor. The estradiol receptor antagonist may comprise Fulvestrant or a pharmaceutically acceptable salt thereof.

Inhibiting the estrogen hormone-induced expression or biologic activity of MMP2 may comprise contacting the smooth muscle cells with an estrogen receptor antagonist in an amount effective to inhibit the estrogen hormone-induced expression or biologic activity of MMP2. The antagonist may comprise Fulvestrant or a pharmaceutically acceptable salt thereof. The antagonist may be present in a composition comprising a pharmaceutically acceptable carrier.

The invention features methods for treating lymphangioleiomyomatosis (LAM). In general, the methods may comprise administering to a subject in need thereof Fulvestrant or a pharmaceutically acceptable salt thereof in an amount effective to treat LAM. The methods may comprise administering to a subject in need thereof a composition comprising a pharmaceutically acceptable carrier and Fulvestrant or a pharmaceutically acceptable salt thereof in an amount effective to treat LAM.

The methods may be used to treat LAM in any subject, with human beings being preferred, and female human beings being highly preferred. The human being may have tuberous sclerosis complex. The human being may have at least one germline mutation in the gene encoding tuberous sclerosis protein 2 (TSC2). The human being may have at least one germline mutation in the gene encoding tuberous sclerosis protein 1 (TSC1). The human being may have at least one germline mutation in the gene encoding TSC1 and at least one germline mutation in the gene encoding TSC2. Non-limiting examples of such mutations are described above. The human being may be a lung transplant patient. A lung transplant may have been because the human being was diagnosed as having LAM.

In some aspects, the methods may further comprise administering to the subject an effective amount of Doxycycline. In some aspects, the methods may further comprise administering to the subject an effective amount of a statin such as simvastatin. In some aspects, the methods may further comprise administering to the subject an effective amount of choloroquine. In some aspects, the methods may further comprise administering to the subject an effective amount of Sirolimus. In some aspects, the methods may further comprise administering to the subject an effective amount of Doxycycline, simvastatin, Sirolimus, or chloroquine.

In some aspects, treating LAM may improve the prognosis of the subject. For example, a prognosis may comprise the patient's probability of survival within about five years. In some aspects, a prognosis may comprise the patient's probability of survival within about ten years. In some aspects, a prognosis may comprise the patient's probability of survival within about fifteen years. In some aspects, a prognosis may comprise the patient's probability of survival within about twenty years.

The invention also features kits for treating lymphangioleiomyomatosis (LAM). The kits generally comprise Fulvestrant or a pharmaceutically acceptable salt thereof in an amount effective to treat LAM or a composition comprising a pharmaceutically acceptable carrier and an amount of Fulvestrant or a pharmaceutically acceptable salt thereof effective to treat LAM, and instructions for using the kit in a method for treating LAM. The kits may further comprise an effective amount of Doxycycline, simvastatin, or chloroquine, or a composition comprising a pharmaceutically acceptable carrier and an effective amount of Doxycycline, simvastatin, Sirolimums, or chloroquine. The method for treating LAM may be any method described or exemplified herein.

For use in any of the methods or kits described herein, Fulvestrant may be formulated as a composition, for example, with a carrier. Compositions may comprise Fulvestrant, or a pharmaceutically acceptable salt thereof. The carrier is preferably a pharmaceutically acceptable carrier. Pharmaceutically acceptable carriers include aqueous vehicles such as water, alcohol (e.g., ethanol or glycol), saline solutions, dextrose solutions, and balanced salt solutions, as well as nonaqueous vehicles such as alcohols and oils, including plant or vegetable-derived oils such as olive oil, cottonseed oil, corn oil, canola oil, sesame oil, and other non-toxic oils. The compositions may comprise one or more pharmaceutically acceptable excipients.

The compositions preferably comprise an effective amount of Fulvestrant or pharmaceutically acceptable salt of Fulvestrant. The compositions may be prepared to provide about 0.05 mg to about 1000 mg of Fulvestrant, or pharmaceutically acceptable salt thereof, though amounts less than about 0.05 mg and more than about 1000 mg may be used. The compositions may comprise about 1 mg to about 500 mg of Fulvestrant, may comprise about 100 mg to about 600 mg of Fulvestrant, may comprise about 10 mg to about 100 mg of Fulvestrant, may comprise about 250 mg to about 500 mg of Fulvestrant, may comprise about 400 mg to about 600 mg of Fulvestrant, may comprise about 100 mg to about 300 mg of Fulvestrant, may comprise about 500 mg to about 750 mg of Fulvestrant, may comprise about 600 to about 800 mg of Fulvestrant and may comprise from about 50 mg to about 250 mg of Fulvestrant, or pharmaceutically acceptable salt thereof. The compositions may comprise about 100 mg of Fulvestrant, may comprise about 200 mg of Fulvestrant, may comprise about 300 mg of Fulvestrant, may comprise about 400 mg of Fulvestrant, may comprise about 500 mg of Fulvestrant, or may comprise about 600 mg of Fulvestrant.

The compositions may be formulated for administration to a subject in any suitable dosage form. The compositions may be formulated for oral, buccal, nasal, transdermal, parenteral, injectable, intravenous, subcutaneous, intramuscular, rectal, or vaginal administrations. The compositions may be formulated in a suitable controlled-release vehicle, with an adjuvant, or as a depot formulation.

Preparations for parenteral administration include sterile solutions ready for injection, sterile dry soluble products ready to be combined with a solvent just prior to use, including hypodermic tablets, sterile suspensions ready for injection, sterile dry insoluble products ready to be combined with a vehicle just prior to use and sterile emulsions.

Solid dosage forms include tablets, pills, powders, bulk powders, capsules, granules, and combinations thereof. Solid dosage forms may be prepared as compressed, chewable lozenges and tablets which may be enteric-coated, sugar coated or film-coated. Solid dosage forms may be hard or encased in soft gelatin, and granules and powders may be provided in non-effervescent or effervescent form. Solid dosage forms may be prepared for dissolution or suspension in a liquid or semi-liquid vehicle prior to administration.

Liquid dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions may be oil-in water or water-in-oil emulsions.

Pharmaceutically acceptable excipients utilized in solid dosage forms include coatings, binders, lubricants, diluents, disintegrating agents, coloring agents, flavoring agents, preservatives, sweeteners, and wetting agents. Enteric-coated tablets, due to their enteric-coating, resist the action of stomach acid and dissolve or disintegrate in the neutral or alkaline intestines. Other examples of coatings include sugar coatings and polymer coatings. Sweetening agents are especially useful in the formation of chewable tablets and lozenges. Pharmaceutically acceptable excipients used in liquid dosage forms includes solvents, suspending agents, dispersing agents, emulsifying agents, surfactants, emollients, coloring agents, flavoring agents, preservatives, and sweeteners.

Non-limiting examples of binders include glucose solution, acacia mucilage, gelatin solution, sucrose and starch paste. Non-limiting examples of lubricants include talc, starch, magnesium or calcium stearate, lycopodium and stearic acid. Non-limiting examples of diluents include lactose, sucrose, starch, kaolin, salt, mannitol and dicalcium phosphate. Non-limiting examples of disintegrating agents include corn starch, potato starch, bentonite, methylcellulose, agar and carboxymethylcellulose. Non-limiting examples of emulsifying agents include gelatin, acacia, tragacanth, bentonite, and surfactants such as polyoxyethylene sorbitan monooleate. Non-limiting examples of suspending agents include sodium carboxymethylcellulose, pectin, tragacanth, veegum and acacia.

Non-limiting examples of coloring agents include any of the approved certified water soluble FD and C dyes, mixtures thereof, and water insoluble FD and D dyes suspended on alumina hydrate. Non-limiting examples of sweetening agents include dextrose, sucrose, fructose, lactose, mannitol and artificial sweetening agents such as saccharin, aspartame, sucralose, acelsulfame potassium, and other artificial sweeteners. Non-limiting examples of flavoring agents include synthetic flavors and natural flavors extracted from plants such as fruits and mints, and synthetic blends of compounds which produce a pleasant sensation. Non-limiting examples of wetting agents include propylene glycol monostearate, sorbitan monooleate, diethylene glycol monolaurate and polyoxyethylene laural ether. Non-limiting examples of enteric-coatings include fatty acids, fats, waxes, shellac, ammoniated shellac and cellulose acetate phthalates. Non-limiting examples of film coatings include hydroxyethylcellulose, sodium carboxymethylcellulose, polyethylene glycol 4000 and cellulose acetate phthalate. Non-limiting examples of preservatives include glycerin, methyl and propylparaben, ethylparaben, butylparaben, isobutylparaben, isopropylparaben, benzylparaben, citrate, benzoic acid, sodium benzoate and alcohol.

Elixirs include clear, sweetened, hydroalcoholic preparations. Pharmaceutically acceptable carriers used in elixirs include solvents. Syrups include concentrated aqueous solutions of a sugar, for example, sucrose, and may contain a preservative. An emulsion is a two-phase system in which one liquid is dispersed throughout another liquid. Pharmaceutically acceptable carriers used in emulsions may include emulsifying agents and preservatives. Suspensions may use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substance used in effervescent granules, to be reconstituted into a liquid oral dosage form, include organic acids and a source of carbon dioxide. Sources of carbon dioxide include sodium bicarbonate and sodium carbonate. Coloring and flavoring agents may be used in all such dosage forms.

Additional excipients that may be included in any dosage forms include, but are not limited to antimicrobial agents, isotonic agents, buffers, antioxidants, local anesthetic agents, sequestering or chelating agents, analgesic agents, antiemetic agents, and other agents to enhance selected characteristics of the formulation.

Antimicrobial agents may be cidal or static, and may be antimicrobial, antifungal, antiparasitic, or antiviral. Non-limiting examples of commonly used antimicrobial agents include phenols or cresols, mercurials, benzyl alcohol, chlorobutanol, methyl and propyl p-hydroxybenzoic acid esters, thimerosal, benzalkonium chloride and benzethonium chloride. Acidic or basic pH may be used for antimicrobial effects in some aspects. Non-limiting examples of isotonic agents include sodium chloride and dextrose. Non-limiting examples of buffers include phosphate and citrate buffers. A non-limiting example of a chelating agent for metal ions is EDTA.

The following examples are provided to describe the invention in greater detail. They are intended to illustrate, not to limit, the invention.

### EXAMPLE 1

### Experimental Methods

Cell culture and reagents. ELT-3 cells (Eker rat uterine leiomyoma-derived smooth muscle cells) were cultured in IIA complete medium supplemented with 15% fetal bovine serum (FBS). Prior to the in vitro experiments, cells were maintained in media supplemented with 10% charcoal-stripped FBS for three days, and then serum-starved for 24 hours in serum-free and phenol red-free medium. 17 beta-estradiol (E₂, 10 nM, Sigma, St. Louis, Mo.), PD98059 (50 μM, Cell Signaling Technology, Danvers, Mass.), or ICI 182780 (1 μM, Biomol) were added to the cells as indicated.

Animal studies. All animal work was performed in accordance with protocols approved by the Institutional Animal Care and Use Committee at the Brigham and Women's Hospital. Female ovariectomized and male CB17-SCID mice, six to eight weeks of age, were purchased from Taconic (Hudson, N.Y.). One week prior to ELT3 cell injection, 17-beta estradiol or placebo pellets (2.5 mg, 90-day release, Innovative Research America, Sarasota, Fla.) were implanted. For xenograft tumor establishment, 2×10⁶ ELT3 cells were bilaterally injected into the rear flanks of the mice. Lung metastases were scored from five-micron H & E stained sections of each lobe by observers blinded to the experimental conditions. Fulvestrant (1 mg/day, intramuscular injection) or Doxycycline (0.7 mg/day, 60-day release, Innovative Research America) treatments were initiated one day post-cell inoculation.

Immunoblotting and antibodies. Cells were rinsed once in ice-cold PBS and lysed in m-PER buffer (50 mM HEPES, pH 7.5, 50 mM NaCl, 5 mM EDTA, 50 mM NaF, 10 mM Na₄P₂O₇, and 1% Triton, Pierce). Lysates were resolved by SDS-PAGE electrophoresis and transferred onto Immobilon P membranes (Millipore, Bedford, Mass.). The following antibodies were used for Western blot analysis: anti-MMP2 (Chemicon), anti-phospho-p42/44 MAPK (T202/Y204), anti-p42/44MAPK, anti-Collagen Type IV (Abnova), anti-Ki67 (BioGenex, San Ramon, Calif.), anti-beta-actin (Sigma), and anti-ERα (Santa Cruz, Calif.). Western blots were developed using horseradish peroxidase-conjugated secondary antibodies and ECL chemiluminescence (Amersham Biosciences, Piscataway, N.J.).

Immunohistochemistry. Sections were deparaffinized, incubated overnight with primary antibodies at 4° C. in a humidified chamber and then rinsed and incubated with biotinylated secondary antibodies for 30 minutes at room temperature. Slides were developed using the Broad Spectrum AEC Histostain®-Plus or Histostain®-Plus kit (Invitrogen), and they were counterstained with Gill's Hematoxylin. TUNEL assay reagent was from Roche. Gelatin zymography. Conditioned media from cultured cells were collected, filtered through 0.2 μM Nalgene filter, and subjected for gel electrophoresis on 10% SDS-PAGE containing 0.1% gelatin (Invitrogen). MMP2 activity was detected according to the manufacture's protocol. Real-time RT-PCR. Total RNA from cultured cells and xenograft tumors was isolated using RNeasy® Mini Kit (Qiagen, Valencia, Calif.). Gene expression was quantified using One-Step qRT-PCR Kits (Invitrogen) in the Applied Biosystems Real-Time PCR System and normalized to beta-actin control amplification.

Statistical analyses. Statistical analyses were performed using Student's t-test when comparing two groups. Results are presented as means+SD (Standard Deviation) of experiments performed in triplicate. Differences were considered significant at p<0.05.

### EXAMPLE 2

### Experimental Results

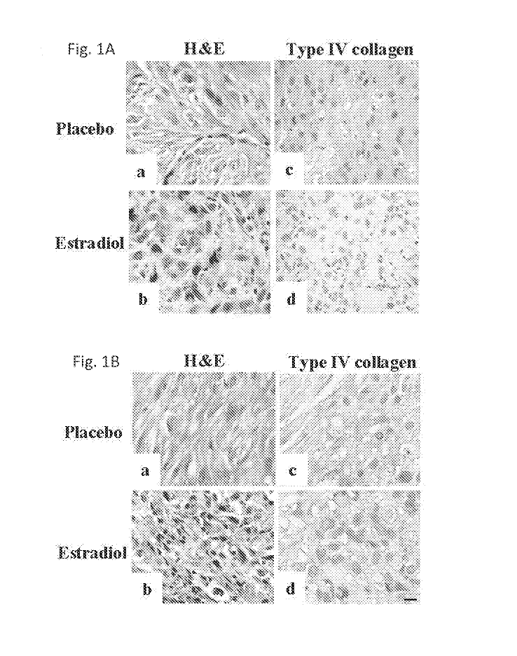

Estradiol reduces ECM organization in female and male mice. It was observed that E₂ promotes a three- to five-fold increase in pulmonary metastasis of ELT3 cells in mice bearing xenograft tumors. Since ECM loss is associated with tumor cell metastasis, morphology of the ECM was examined in primary tumors from ovariectomized female mice and male mice treated with placebo or estradiol. In xenograft tumors from placebo-treated animals, the ECM organization was well-maintained in female and male mice FIG. 1A-a and FIG. 1B-a. In contrast, the xenograft tumors from estradiol-treated animals exhibited disrupted ECM network in female and male mice (FIG. 1A-b and FIG. 1B-b). This estradiol-induced ECM disruption was associated with a marked reduction of type IV collagen in female and male mice (FIG. 1A-d and FIG. 1B-d).

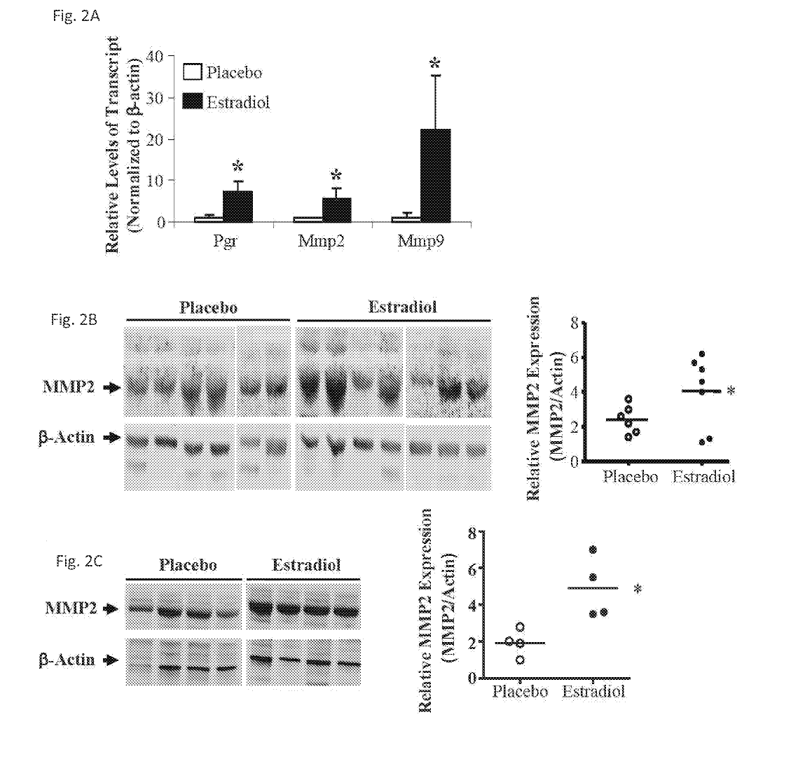

Estradiol increases MMP2 accumulation in tumor cells in vivo. Type IV collagen is degraded, in part, by MMPs, particularly by MMP2. To examine levels of MMP2 in xenograft tumors of rat uterine leiomyoma-derived ELT3 cells from a metastatic model of LAM, MMP2 transcript levels were first measured by real-time RT-PCR. It was observed that xenograft tumors from estradiol-treated female mice expressed higher levels of MMP2 and MMP9 compared with size-matched tumors from placebo-treated mice, by 5-fold and 28-fold, respectively (p<0.05, n=4, FIG. 2A). The level of progesterone receptor (PgR), an estrogen-responsive gene, was increased in tumors from estradiol-treated mice compared with placebo-treated, by 4-fold. MMP2 protein levels were also examined by immunoblotting. The xenograft tumors from estradiol-treated female mice had higher level of MMP2 by 1.8-fold (p<0.05, n=7, FIG. 2B), and by 2.5-fold in estradiol-treated male mice (p<0.05, n=4, FIG. 2C).

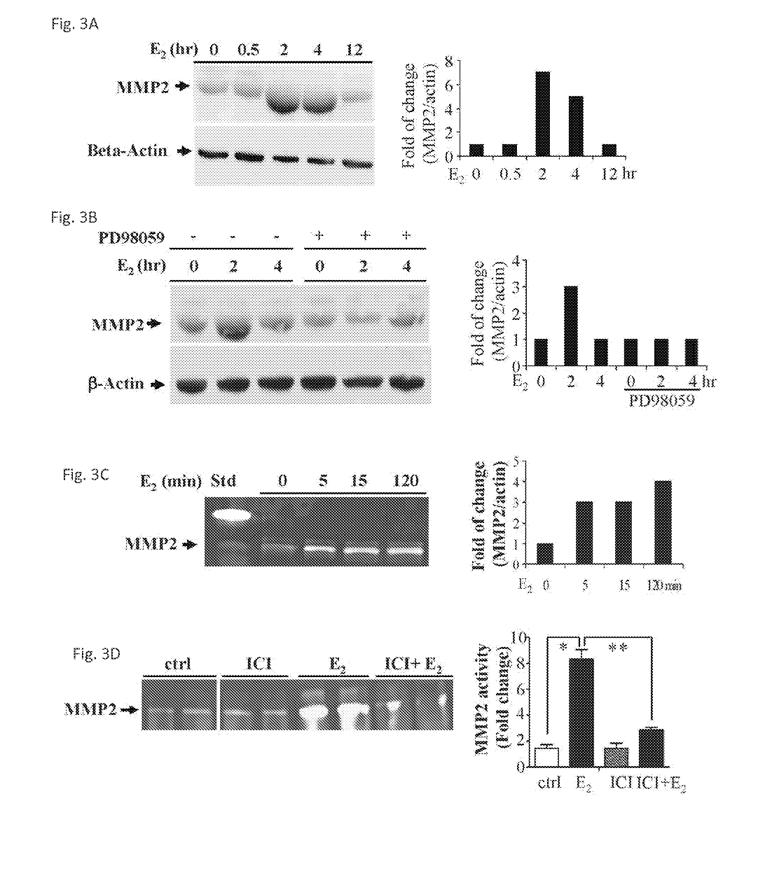

Estradiol increases MMP2 expression and activity in tuberin-null ELT3 cells in vitro. To confirm the in vivo findings, cultured ELT3 cells were treated with estradiol and levels of MMP2 accumulation were analyzed using immunoblotting. Estradiol increased MMP2 accumulation by five to seven-fold within two to four hours of treatment (FIG. 3A). Preincubation with the MEK inhibitor PD98059 for 30 minutes strongly blocked estradiol's enhancement of MMP2 accumulation after two hours of treatment (FIG. 3B).

To identify the earliest time points at which estradiol exerts an effect on the activity of MMP2 in tuberin-null ELT3 cells, MMP2 activity was measured using gelatin zymography. Within five minutes of estradiol's stimulation, active MMP2 level was increased by three-fold. This estradiol-stimulated MMP2 activity was sustained throughout the 120 minutes stimulation (FIG. 3C).

To further define the kinetics of estradiol-stimulated MMP2 activity, ELT3 cells were stimulated with estradiol for 24 hours. MMP2 activity increased by eight-fold (FIG. 3D). To determine whether estradiol-stimulated MMP2 activity is mediated by the estrogen receptor, ELT3 cells were treated with the estrogen receptor antagonist ICI182780 for 4 hours, followed by estradiol stimulation. Pretreatment of ELT3 cells with ICI182780 almost completely blocked estradiol-stimulated MMP2 activity (p<0.01) (FIG. 3D).

Doxycycline treatment does not block estrogen-induced pulmonary metastasis of TSC2-deficient cells. In women with LAM, Doxycycline has been proposed as a therapeutic agent based on its ability to improve lung function. Thus, whether Doxycycline inhibits estradiol-induced xenograft tumor growth and metastases was assessed.

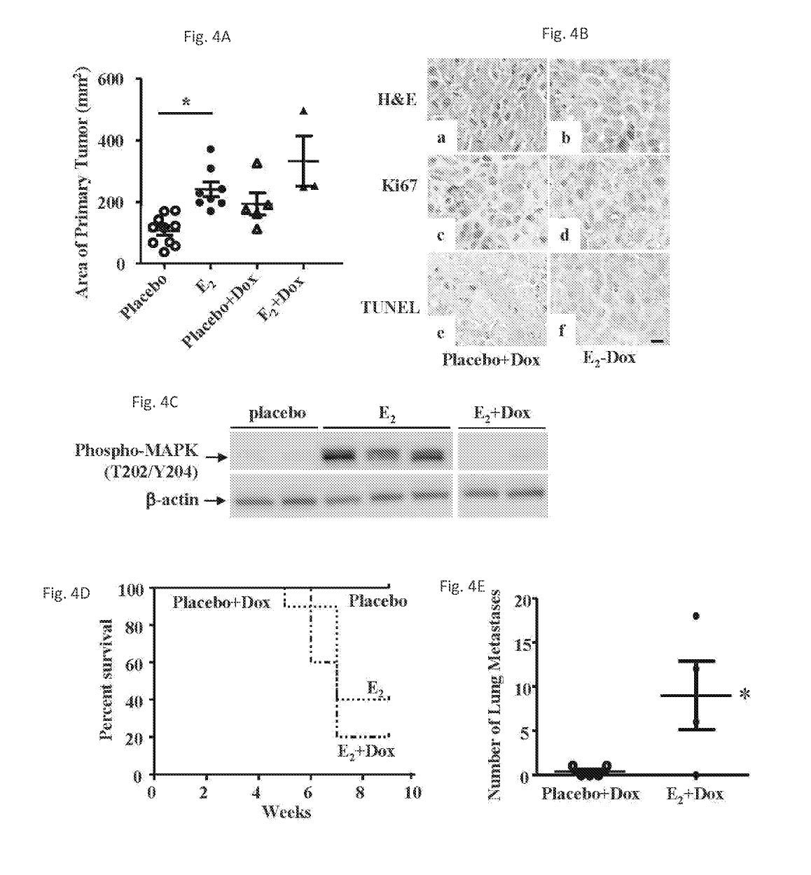

Doxycycline (60-day slow-releasing pellet, 0.7 mg/day) was administered beginning one day post-subcutaneous cell inoculation of ELT3 cells. The mTORC1 inhibitor RAD001 given on this schedule (one day prior to cell inoculation) completely blocked subcutaneous tumor development and lung metastasis. At post-inoculation week eight, estrogen-treated mice had a mean tumor area of 251±65 mm², whereas placebo-treated mice had a mean tumor area of 107±47 mm² (p=0.0005) (FIG. 4A). Doxycycline plus estradiol-treated mice had a mean tumor area of 333±142 mm², whereas Doxycycline plus placebo-treated mice had a mean tumor area of 193±80 mm² (p=0.22, Dox plus E₂ vs. Dox plus Placebo; p=0.43, Dox plus E₂ vs. E₂) (FIG. 4A).

In the xenograft tumors from Doxycycline plus placebo-treated animals, ECM organization was well maintained (FIG. 4B-a). In contrast, the xenograft tumors from Doxycycline plus estradiol-treated animals exhibited disrupted ECM network (FIG. 4B-b) similar to that of the estradiol-treated animals in FIG. 1. The proliferative potential of ELT3 xenograft tumor cells was measured using Ki-67 immunoreactivity. Doxycycline treatment did not affect cell proliferation (FIG. 4B-c, d) or the levels of cell death determined by TUNEL staining (FIG. 4B-e, f), in xenograft tumors from estradiol-treated mice. Despite the lack of evidence of an impact on ECM, cell proliferation, or apoptosis, tumors from mice treated with estradiol plus Doxycycline had lower levels of phospho-p42/44 MAPK (FIG. 4C). Moreover, estradiol-treated mice bearing xenograft tumors had reduced overall survival than placebo-treated mice, which was not rescued by Doxycycline treatment (FIG. 4D).

Pulmonary metastases were identified in five of nine E₂-treated mice (56%), with an average of 10 metastases/mouse (range 4-37). Four of five Doxycycline plus estradiol-treated mice (80%) developed lung metastases with an average of 9 metastases/mouse (range 6-18) (FIG. 4E). Together, these data indicate that Doxycycline (at the dose used in this study) does not inhibit tumor progression or lung metastasis in this model of LAM.

The estrogen receptor antagonist Fulvestrant normalizes estrogen-promoted ECM disruption in vivo. To further investigate the effect of inhibiting the estrogen receptor pathway on estradiol-induced ECM alteration in vivo, the ER antagonist Fulvestrant was used. Fulvestrant is FDA-approved for breast cancer treatment.

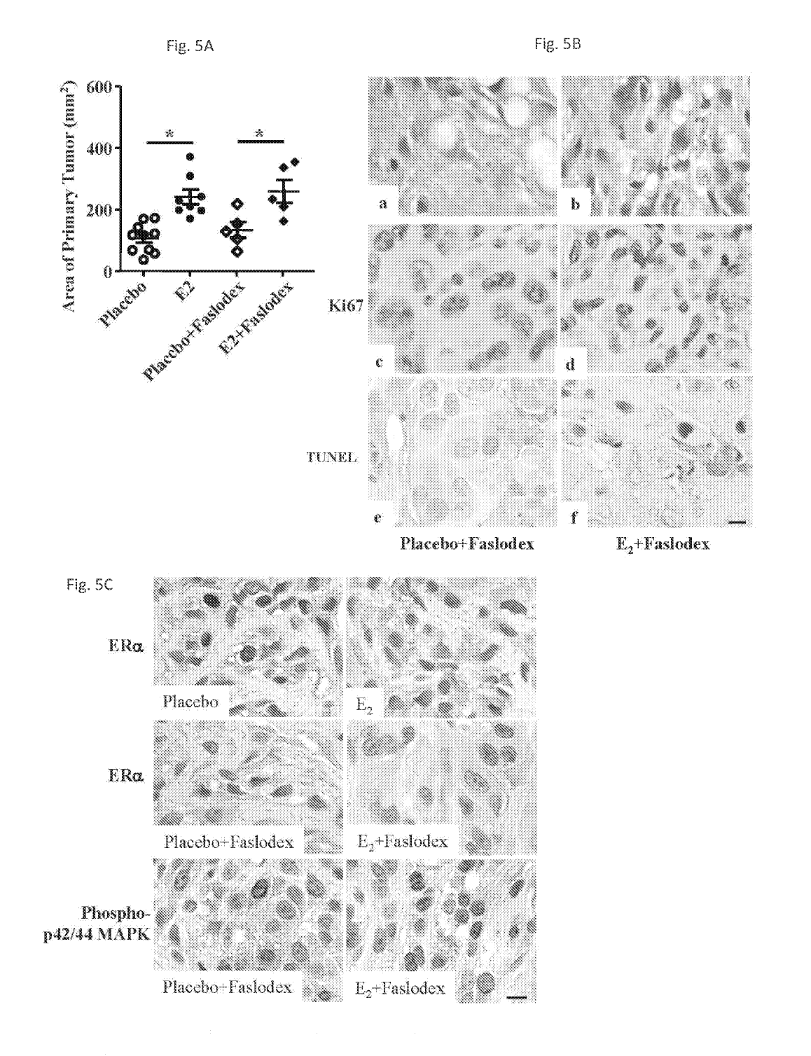

Beginning one day post-subcutaneous inoculation of ELT3 cells, animals, implanted with either placebo or estrogen pellets, were treated with Fulvestrant (1 mg/day intramuscular). At post-inoculation week eight, estradiol-treated mice had a mean tumor area of 251±65 mm², whereas placebo-treated mice had a mean tumor area of 107±47 mm² (p<0.05) (FIG. 5A). Fulvestrant plus estradiol-treated mice had a mean tumor area of 259±83 mm² (p=0.03, Ful plus E₂ vs. Ful plus Placebo; p=0.86, Ful plus E₂ vs. E₂) (FIG. 5A).

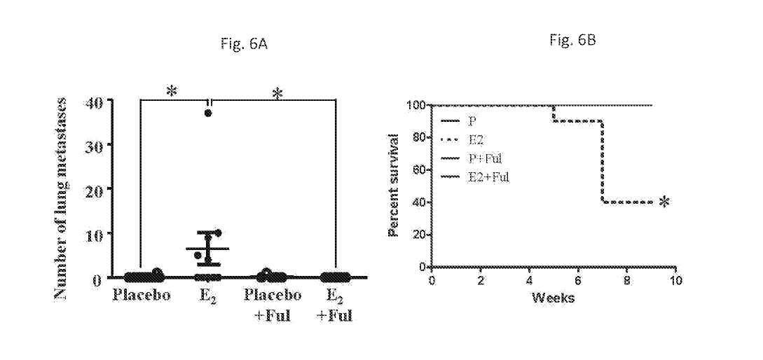

Xenograft tumors from Fulvestrant plus placebo-treated animals exhibited dense bundles of collagen fibers (FIG. 5B-a). The xenograft tumors from Fulvestrant plus estradiol-treated animals exhibited a well-maintained ECM network (FIG. 5B-b) similar to the placebo-treated animals in FIG. 1. Fulvestrant treatment did not affect the Ki-67 immunoreactivity (FIG. 5B-c, d) or alter the levels of TUNEL positive cells (FIG. 5B-e, f) in xenograft tumors from in estradiol plus Fulvestrant-treated mice. Despite this lack of evidence of an impact on cell proliferation and apoptosis, tumors from mice treated with estradiol plus Fulvestrant had lower levels of ERα and phospho-p42/44 MAPK (FIG. 5C), suggesting that Fulvestrant was effective in vivo. Fulvestrant completely blocked estradiol-promoted lung metastases (p=0.046, FIG. 6A) and significantly increased the survival of E₂-treated mice bearing xenograft tumors (p<0.05, FIG. 6B).

### EXAMPLE 3

### Summary

It was previously found that estradiol promotes the metastasis of TSC2-deficient ELT3 tumors. ELT3 cells were used as a model of LAM because they are smooth muscle-derived, express ERα and progesterone receptor, and respond to estradiol stimulation in vitro and in vivo. The data in these examples show that estradiol alters the architecture of ELT3 xenograft tumors. This was associated with decreased type IV collagen and increased cellular MMP2. A marked increase of MMP2 and MMP9 transcript levels in xenograft tumors from estradiol-treated mice was also found. In vitro, estradiol enhanced the expression, accumulation and activity of MMP2 in a MEK1/2-dependent manner. In mice bearing xenograft tumors, administration of the MMP inhibitor Doxycycline did not affect the growth of xenograft tumors or estradiol-induced lung metastasis, although it did inhibit the phosphorylation of MAPK. In contrast, the estrogen receptor antagonist Fulvestrant normalized extracellular matrix organization, inhibited estradiol-promoted lung metastases, and enhanced the survival of estradiol-treated mice bearing xenograft tumors.

Metastasis is a multi-step process, and there are several distinct mechanisms through which steroid hormones may promote the pathogenesis of LAM. Without intending to be limited to any particular theory or mechanism of action, it is believed that these data support a model in which estradiol induces disruption of the ECM of LAM nodules and reduces levels of type IV collagen, thereby promoting the dissemination of LAM cells. This is in agreement with previous finding that showed estradiol increased the level of circulating disseminated tumor cells, and the identification of circulating LAM cells in the blood, urine and chylous fluid of LAM patients.

Degradation of elastic fibers has been observed in regions of smooth muscle cells within LAM nodules and type IV collagen has been found to colocalize with MMP2 in LAM nodules. Furthermore, it has been previously observed that tuberin-null LAM associated angiomyolipoma-derived cells express higher MMP2 transcript levels than TSC2-reexpressing cells. The data indicate that not only the MMP2 levels are increased in TSC2-deficient cells, but estradiol further enhances MMP2 activity. These in vitro and in vivo findings support the notion that estradiol synergizes with TSC2 loss to enhance MMP2 expression.

Doxycycline, a tetracycline derivative, nonselectively inhibits MMPs by causing conformational changes and loss of enzymatic activity. In the studies described in the preceding Examples, Doxycycline treatment did not affect the growth of xenograft tumors or the estradiol-promoted lung metastasis of ETL3 cells.

In contrast to Doxycycline, which did not affect estradiol-induced lung metastasis in the model, the estrogen receptor antagonist Fulvestrant completely blocked estradiol-promoted lung metastasis and enhanced the survival of mice carrying xenograft tumors. Fulvestrant is an estrogen receptor antagonist which disrupts ligand binding, receptor dimerization and nuclear translocation, and accelerates receptor degradation. The data suggest that Fulvestrant is a potential novel therapeutic agent for LAM. Fulvestrant may have potential advantages in LAM, compared to tamoxifen and other selective ER modulators (SERMs), because it is believed that it does not have agonist activity.

We claim:

1. A method for treating lymphangioleiomyomatosis (LAM), comprising administering to a subject in need thereof Fulvestrant or a pharmaceutically acceptable salt thereof in an amount effective to treat LAM.

2. The method of claim 1, wherein the Fulvestrant or pharmaceutically acceptable salt thereof is comprised in a composition comprising a pharmaceutically acceptable carrier.

3. The method of claim 1, further comprising administering to the subject an effective amount of Doxycycline.

4. The method of claim 1, wherein the subject is a human being.

5. The method of claim 4, wherein the human being has tuberous sclerosis complex-associated LAM or sporadic LAM.

6. The method of claim 4, wherein the human being has at least one germline mutation in the gene encoding tuberous sclerosis protein 2 (TSC2) or in the gene encoding tuberous sclerosis protein 1 (TSC1).

7. A method for inhibiting estrogen hormone-induced pulmonary metastasis of smooth muscle cells capable of pulmonary metastasis, comprising antagonizing an estrogen receptor on the smooth muscle cells such that pulmonary metastasis of the cells is inhibited.

8. The method of claim 7, wherein the smooth muscle cells have at least one mutation in the gene encoding tuberous sclerosis protein 2 (TSC2) or have at least one mutation in the gene encoding tuberous sclerosis protein 1 (TSC1),

9. The method of claim 7, wherein antagonizing the estrogen receptor comprises contacting the smooth muscle cells with an estrogen receptor antagonist in an amount effective to antagonize the estrogen receptor.

10. The method of claim 7, wherein the estrogen receptor is the estradiol receptor.

11. The method of claim 8, wherein the estrogen receptor antagonist is an estradiol receptor antagonist.

12. The method of claim 11, wherein the estradiol receptor antagonist comprises Fulvestrant or a pharmaceutically acceptable salt thereof.

13. The method of claim 7, wherein antagonizing the estrogen receptor comprises administering to a subject in need thereof an estrogen receptor antagonist in an amount effective to antagonize the estrogen receptor.

14. The method of claim 13, wherein the estrogen receptor antagonist is an estradiol receptor antagonist.

15. The method of claim 14, wherein the estradiol receptor antagonist comprises Fulvestrant or a pharmaceutically acceptable salt thereof.

16. The method of claim 13, wherein the subject is a human being.

17. The method of claim 16, wherein the human being has tuberous sclerosis complex-associated lymphangioleiomyomatosis or sporadic lymphangioleiomyomatosis.

18. The method of claim 16, wherein the human being has at least one germline mutation in the gene encoding tuberous sclerosis protein 2 (TSC2) or in the gene encoding tuberous sclerosis protein 1 (TSC1).

19. The method of claim 7, wherein the smooth muscle cells express one or more of melanoma glycoprotein 100 and the HMB 45 antigen.

20. A kit for treating lymphangioleiomyomatosis (LAM), comprising a composition comprising a pharmaceutically acceptable carrier and an amount of Fulvestrant or a pharmaceutically acceptable salt thereof effective to treat LAM, and instructions for using the kit in a method for treating LAM.
